(19) United States
(12) Patent Application Publication
(10) Pub. No.: US 2004/0106891 A1
(43) Pub. Date: Jun. 3, 2004

(54) LOCALIZING NEEDLE WITH FLUID DELIVERY

(75) Inventors: James J. Langan, Rockford, IL (US); Ryan L. Goosen, Coopersville, MI (US); Steven E. Field, Grand Rapids, MI (US)

Correspondence Address: MCGARRY BAIR PC, 171 MONROE AVENUE, N.W., SUITE 600, GRAND RAPIDS, MI, 49503, US

(73) Assignee: INRAD, INC., Kentwood, MI (US)

(21) Appl. No.: 10/604,948

(22) Filed: Aug. 28, 2003

Related U.S. Application Data
(60) Provisional application No. 60/319,513, filed on Aug. 30, 2002.

(51) Int. Cl. A61N 1/30

(52) U.S. Cl. 604/19; 600/424

(57) ABSTRACT

A localizing needle comprises a barbed localizing wire fixedly sheathed within a flexible hollow infusion tube for delivering fluids to surrounding tissue through a plurality of spaced infusion ports. Echogenic and/or radiopaque markers are provided at selected intervals along the infusion tube. A helical wire bead at a distal end of the tube anchors the wire to the tube.

### CROSS REFERENCE TO RELATED APPLICATIONS

This application claims the benefit U.S. Provisional Patent Application Serial No. 60/319,513, filed Aug. 30, 2003, entitled “LOCALIZING NEEDLE WITH LIQUID DELIVERY.

### BACKGROUND OF INVENTION

1. Field of the Invention

The invention relates to a localizing needle. In one aspect, the invention relates to a localizing needle comprising a localizing wire fixedly attached to a fluid infusion tube. In another aspect, the invention relates to a localizing needle having positional markings for establishing the position of the needle through non-invasive imaging techniques. In another aspect, the invention relates to a localizing needle adapted for delivery of fluids to a biopsy location.

2. Description of the Related Art

It is frequently necessary to sample or remove tissue from humans, particularly in the diagnosis and treatment of cancerous or pre-cancerous conditions. In the case of suspected cancer, particularly cancer of the breast, early detection and diagnosis is critical to the success of the patient's treatment and recovery. Various techniques are available to aid in detection and diagnosis, including physical examination and mammography. When a condition is detected that suggests the possibility of cancer, a biopsy must be performed to obtain tissue samples for a complete diagnosis. A biopsy is frequently performed through an open surgical procedure in which an incision is made to the tissue of interest, which can then be visually examined and excised for further evaluation or as a component of treatment.

It is also frequently desirable to infuse the tissue of interest with a fluid, such as a dye, radioisotope, or other diagnostic or therapeutic fluid. In particular, dyes and radioisotopes are used as diagnostic aids and to assist the practitioner in determining the aerial extent of the biopsy. For example, once the tissue of interest has been located, a dye is injected and its migration through the tissue is monitored. In the case of suspected breast cancers, the dye migrates to lymph nodes which drain the breast tissue. Typically, a single lymph node, called a “sentinel node,” will be the lymph node to which the dye first migrates and which in turn drains to other lymph nodes. If the cancer has spread beyond the tissue of interest, the sentinel node will be the first lymph node in which the cancer will be detected. Thus, the sentinel node is typically evaluated, usually by performing a biopsy of the sentinel node, and, if cancer is not present, no further lymph system evaluation is performed. Thus, saving the patient the additional trauma of subsequent biopsies. Conversely, if cancer is detected in the sentinel node, then further evaluation of the lymph system is performed. Consequently, identification of the sentinel node is an important aspect of breast cancer diagnosis and treatment.

Previous methods for determining the sentinel node included injecting a dye or radioisotope the a hollow localizing wire as described in U.S. Pat. No. 6,261,240. While this product appears to function as intended, it is relatively expensive to manufacture. The relative expense to manufacture appears related to the desire to keep the wire is fairly small to minimize the trauma to the tissue during insertion and the difficulty of hollowing the interior of such a small solid wire. One method of hollowing the solid wire is to use a laser drilling technique, which is fairly expensive. Since most of the devices used to locate the sentinel node are disposable for health and sanitary reasons, there is a strong desire to reduce the cost of these devices.

Another disadvantage of the hollow localizing wire is that it can be cut during the excising of the biopsy. If the wire is cut, a more invasive procedure must be done to remove the portion of the wire within the tissue.

### SUMMARY OF INVENTION

A medical device for injecting fluid into a tissue at a selected location in the tissue comprises an infusion tube having distal and proximal ends and defining a lumen with an outlet for releasing fluid from the infusion tube, with the outlet being proximate the distal end, and a localizing wire having an anchor fixedly mounted to the infusion tube and located proximate to the distal end, wherein the infusion tube is positioned to deliver fluid to the selected location by inserting the distal end of the infusion tube into the tissue such that fluid released through the outlet will flow to the selected location and the anchor engages the tissue to fix the position of the infusion tube.

The infusion tube outlet can comprise multiple ports extending from the lumen to the exterior of the infusion tube. The multiple ports can be arranged in set and radially spaced about the infusion tube. The infusion tube can comprise multiple lumens with the wire extending through one of the multiple lumens.

The localizing wire can be mounted to the exterior or interior of the infusion tube. Preferably, the localizing wire extends coaxially through the lumen of the infusion tube. The anchor is preferably releasable to permit the repositioning of the infusion tube after it is anchored, and can be formed from a flexible hook. Alternatively, the localizing wire can comprise a fiber optic thread with a hook mounted to the fiber optic thread.

Imaging markers can be provided on at least one of the infusion tube and the localizing wire. The markers can be of any suitable type and capable of being imaged by any imaging technique. For example, the markers can be echogenic, radiopaque, magnetic resonance compatible, or a combination thereof. The markers can include physical markings on either of both of the infusion tube and localizing wire. The markers could include filling at least a portion of one of the lumens in a multi-lumen configuration with an imagable material. The markers can include items mounted to the infusion tube/localizing wire, such as a coil for example. Alternatively, the imaging markers can comprise a label imprinted with ink, which can comprise tungsten.

The infusion tube can further comprise an inlet for introducing fluid into the hollow interior, and a syringe fluidly connected to the inlet to introduce fluid into the lumen from the syringe. The syringe can be removably coupled to the inlet.

The medical device can further comprise a cannula for locating the infusion tube into the tissue, having a lumen sized to receive the infusion tube, wherein the infusion tube is received within the cannula lumen, the cannula is inserted into the tissue, and a portion of the infusion tube is exposed to the tissue from the cannula. The medical device can further comprise a syringe fluidly coupled to the infusion tube lumen for the delivery of the fluid to the infusion tube, with a releasable connector, such as a Luer-lock connector, for releasably connecting the syringe to the infusion tube, and the releasable connector is connected to the infusion tube with a compression fitting.

In another embodiment, the invention relates to a method of injecting a fluid into a tissue mass using a localizing needle comprising an infusion tube defining a lumen, a localizing wire fixedly mounted to the infusion tube, and at least one imaging marker located on the infusion tube or localizing wire. The method comprises the steps of: inserting the infusion tube and localizing wire into tissue mass; locating the infusion tube at a predetermined location within the tissue mass by imaging the imaging marker; anchoring the localizing wire at the predetermined location to fix the position of the infusion tube within the tissue mass, and delivering a fluid to the tissue mass through the lumen.

The locating of the infusion tube can comprise locating the infusion tube near a biopsy site in the tissue mass. The delivering of fluid can comprise delivering fluid through at least one port in the infusion tube. The infusion tube can be located near the biopsy site such that the fluid exiting the at least one port flows to the biopsy site.

The method can further comprise the step of detecting the movement of the fluid from the biopsy site to another location. Preferably, the biopsy site is located within a human breast and the another location is a node of the human lymphatic system.

The inserting step can comprise inserting the infusion tube and localizing wire into a cannula. The cannula can be inserted into the tissue prior to or before the insertion of the infusion tube and localizing wire into the cannula. The cannula can be withdrawn from the tissue mass after the locating of the infusion tube.

The method can also comprise the step of detecting the movement of the fluid to a second tissue mass. Preferably, the tissue mass is a human breast and the second tissue mass is a node of the human lymphatic system.

### BRIEF DESCRIPTION OF DRAWINGS

In the drawings:

FIG. 1 is an exploded view of a localizing needle according to the invention and including a localization wire and infusion tube in combination with a cannula for inserting the localizing needle into tissue and a syringe assembly for delivering fluids to the localizing needle.

FIG. 2 is an assembled view of the localizing needle of FIG. 1 inserted into the cannula.

FIG. 3 is a perspective view of a part of the localizing needle of FIG. 1 showing the localization wire with an anchor, a mounting bead, and a plurality of echogenic and/or radiopaque markers comprising a first embodiment of the invention spaced along a hollow tube comprising a part of the localizing needle for locating the needle within the tissue.

FIG. 4 is a sectional view taken along line 4-4 of FIG. 1 and illustrating the spatial relationship between the localization wire and the infusion tube which defines a fluid delivery channel.

FIG. 5 is a perspective view similar to FIG. 3 showing alternative echogenic and/or radiopaque markers, and infusion apertures at spaced intervals along the tube.

FIG. 6 is a perspective view similar to FIG. 3 showing alternative echogenic and/or radiopaque markers.

FIG. 7 is a perspective view similar to FIG. 3 showing alternative echogenic and/or radiopaque markers.

FIG. 8 is a perspective view similar FIG. 3 showing alternative infusion ports in the form of slits at spaced intervals along the tube.

FIG. 9 is a perspective view of a part of the tube of FIG. 3 showing an alternative connection between the localization wire and the infusion tube.

FIG. 10 is a perspective view of an alternative embodiment of the localizing needle of FIG. 2 in which the barb is replaced by a repositionable hook.

FIG. 11 is a sectional view similar to FIG. 9 showing an alternative attachment of the wire to the tube.

FIG. 12 is a sectional view similar to FIG. 9 showing an alternative embodiment of the localizing needle of FIG. 2 comprising a plurality of lumens.

FIG. 13 is a perspective view of an alternative embodiment of the localizing needle of FIG. 2 in which the wire is replaced by a fiber-optic thread.

FIG. 14 is a partially cutaway perspective view of an alternative embodiment of the localizing needle of FIG. 2 comprising an infusion tip attached to the tube.

FIG. 15 is a schematic view of a portion of a breast having a tumor and lymph nodes and showing the insertion of the cannula with a localizing needle into the region of the tumor.

FIG. 16 is a schematic view similar to FIG. 15 of the withdrawal of the cannula leaving the localizing needle anchored in the region of the tumor.

FIG. 17 is a schematic view similar to FIG. 15 of the localizing needle attached to the syringe assembly and injected dye migrating through breast tissue from the region of the tumor to the lymph nodes.

### DETAILED DESCRIPTION

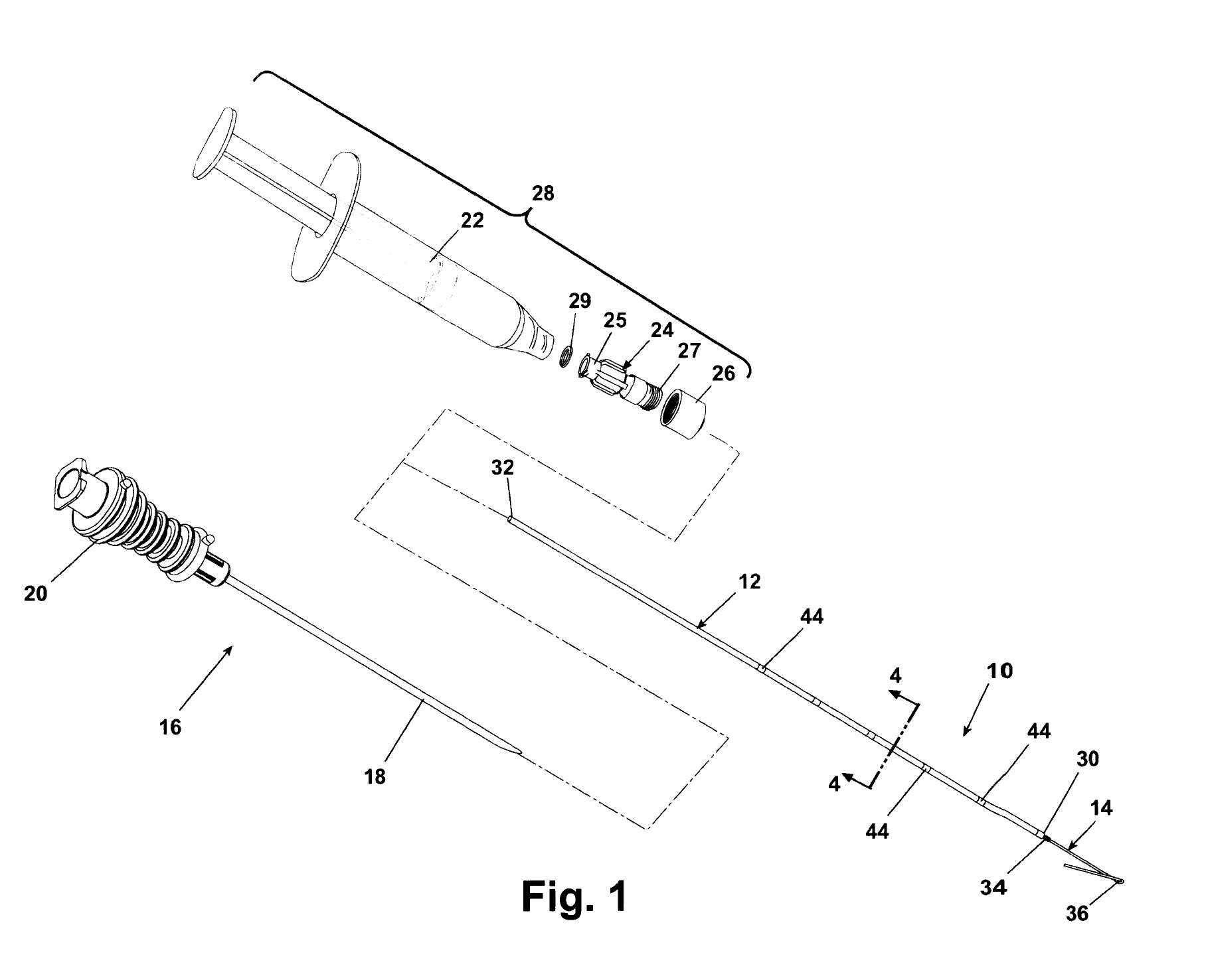

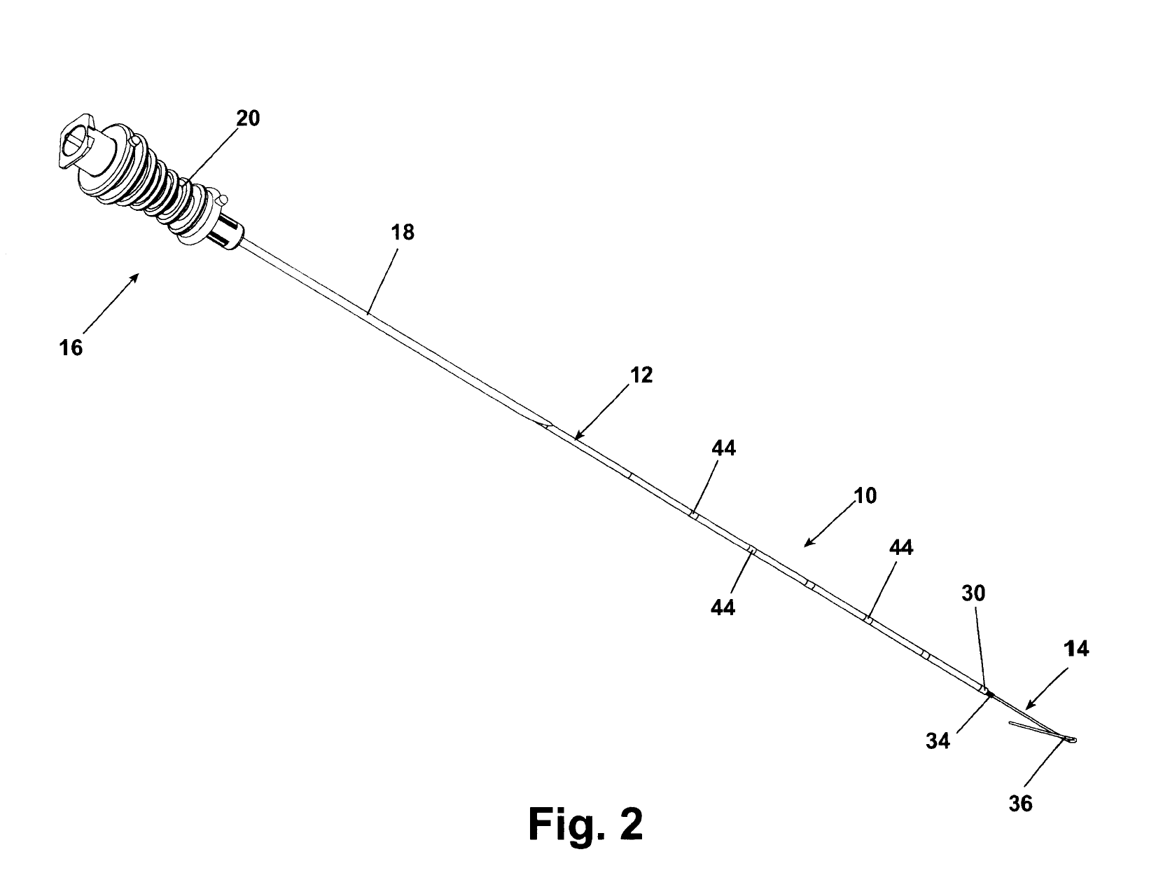

FIGS. 1-3 illustrate a first embodiment of a medical device, shown for illustrative purposes as a localizing needle 10, according to the invention comprising a flexible conduit, such as a flexible infusion tube 12, and an anchoring device, such as a localizing wire 14. A generally conventional cannula 16 having a lumen extending longitudinally therethrough is provided for insertion of the localizing needle 10 into human or animal tissue, and a generally conventional syringe assembly 28 delivers fluids to the infusion tube 12. The cannula 16 comprises a needle 18 and a hub 20 adapted for operable communication with an insertion device.

Referring to FIGS. 1-4, the infusion tube 12 has a distal end 30 and a proximal end 32 and is preferably a thin, flexible, hollow conduit defining a lumen. The tube 12 is preferably made of clear polymeric material having a tube wall 38 of circular cross-section sheathing the localizing wire 14 extending coaxially therethrough to form an annular duct 13 between the tube wall 38 and the localizing wire 14. A suitable material for the infusion tube 12 is a polyurethane, such as Tecothane manufactured by Thermedics Polymer Products of Woburn, Mass.

The localizing wire 14 preferably comprises a high-strength flexible metallic wire of suitable strength and flexibility for the purposes described herein, such as stainless steel, titanium, or a nickel-titanium alloy, such as Nitinol, which has shape memory characteristics. The localizing wire 14 can be provided in selectively varying lengths suitable for the purposes described herein.

A retainer in the form of an anchoring bead 34 is used to fixedly retain the wire 14 to the infusion tube 12. The anchoring bead 34 comprises a helical coil, preferably fabricated of stainless steel, adapted to be in circumferential communication with the wire 14 and partially inserted in the annular duct 13 at the distal end 30. The bead 34 is fixedly attached to the wire 14 and the tube 12 sufficiently to prevent the wire 14 from moving inwardly or outwardly of the tube 12, such as by an ultraviolet-cured adhesive, ultrasonic welding, or other suitable adhesive or method.

To further aid in fixing the wire 14 to the tube 12, the bead 34 can have an outer diameter that is slightly greater than the inner diameter of the tube 12, resulting in a friction-fit or press-fit between the bead 34 and the tube 12.

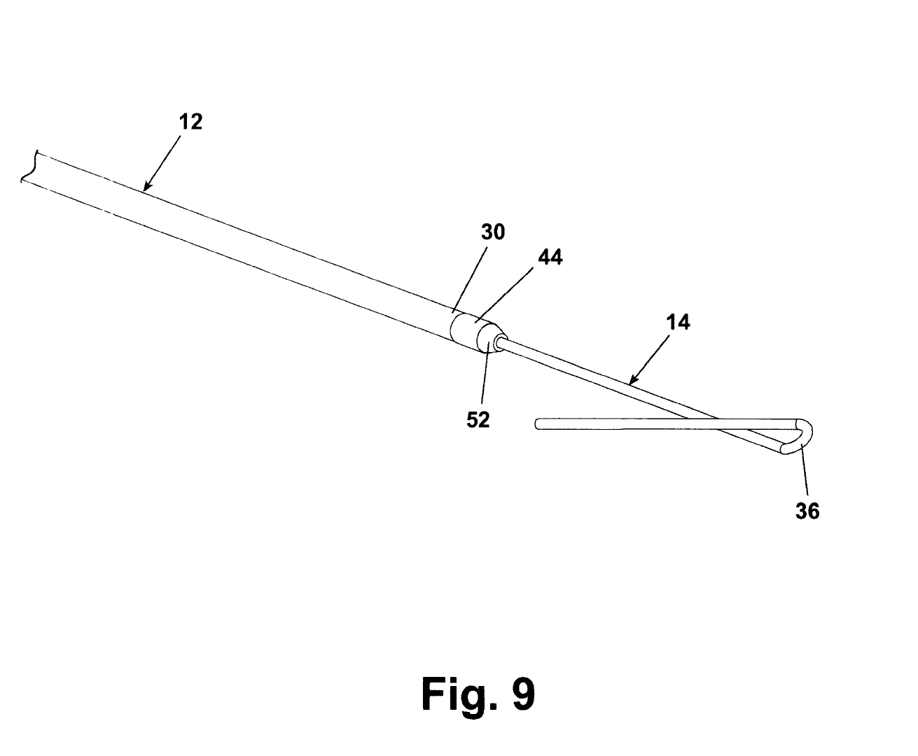

As shown in FIG. 9, an alternative to the bead 34 for fixing the wire 14 to the infusion tube 12 comprises a tapered anchoring cone 52. The anchoring cone 52 is a generally truncated, cone-shaped body which can comprise a separate element to be inserted into the tube 12, or the distal end 30 of the tube 12 can be fabricated with the anchoring cone 52 integral thereto. The wire 14 is then fixedly attached to the anchoring cone 52 by an adhesive or other suitable method.

The retainer could be formed by press-fitting the wire 14 through the tube 12, preferably only at the distal end 30. Such a construction would require the distal end 30 to have a reduced diameter, the wire to have an enlarged diameter at the distal end 30, or a combination of both. Otherwise, the wire 14 would fill the lumen of the tube 12 and eliminate the conduit. The press-fit should be able to resist the relative longitudinal movement of the wire 14 and the tube 12, like the previously described retainers.

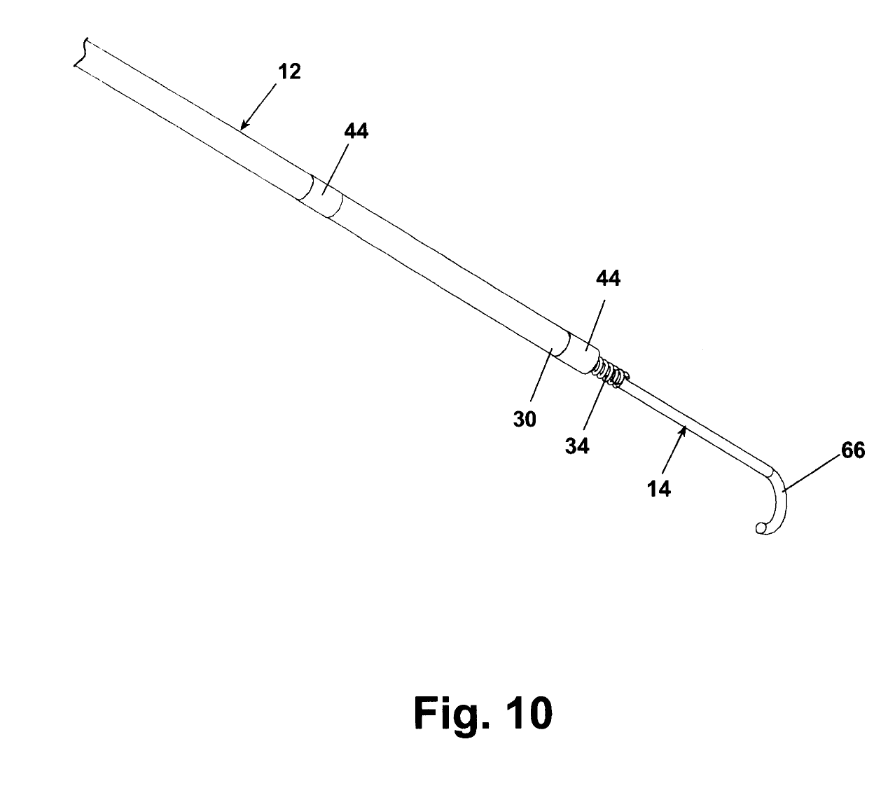

Returning to FIGS. 1-4, the wire 14 is bent into a generally conventional barb 36 to form an anchor for anchoring the localizing needle 10 in the tissue, and preventing the inadvertent movement of the needle 10 after emplacement. As shown in FIG. 10, the wire 14 can also be provided with a repositionable hook 66 of a type well-known in the art instead of the barb 36 to more readily enable the repositioning of the localizing needle 10 after emplacement.

The infusion tube 12 can be provided with a plurality of generally conventional markers 44 having imaging properties, such as echogenic and/or radiopaque properties, for locating the position of the localizing needle 10 within the tissue through standard imaging systems, such as ultrasound, radiographs, magnetic resonance imaging (MRI). The markers 44 can be rings or other structures affixed to the infusion tube 12. The markers could also be structures formed in the surface of the infusion tube 12. For example, the markers could be convolutions or cross-hatched areas formed in the infusion tube.

The markers can also comprise a plurality of marker beads 46 comprising helical coils inserted in the annular duct 13 at preselected intervals. Helical coils fabricated of a ferrous material will have echogenic and radiopaque properties. Helical coils fabricated of a non-magnetic material, such as stainless steel, will be MRI-compatible. The marker beads 46 are fixedly attached to the tube wall 38 such as by an adhesive or other suitable method. The wire 14 is slidably received within the bead 46. The use of a helical coil enables fluid delivered through the annulus 13 to migrate without interruption past the beads 46. If an adhesive is used to attach the bead 46 to the tube 12, the adhesive should not extend from the wire 14 to the tube 12 since fluid passing through the duct 13 would be blocked, negating the fluid delivery function of the invention. The marker beads 46 can be identical to the mounting bead 34. Thus, the marker beads and mounting bead could serve the dual function of mounting the wire 14 to the infusion tube 12 and as an imaging marker.

The markers 44, 46 are preferably located at predetermined distances along the tube 12. A preferred distance is every 2 cm on center. However, any preferred or desired separation distance can be used. The separation distances also need not be identical. For example, the distances can be sequential multiples of each other.

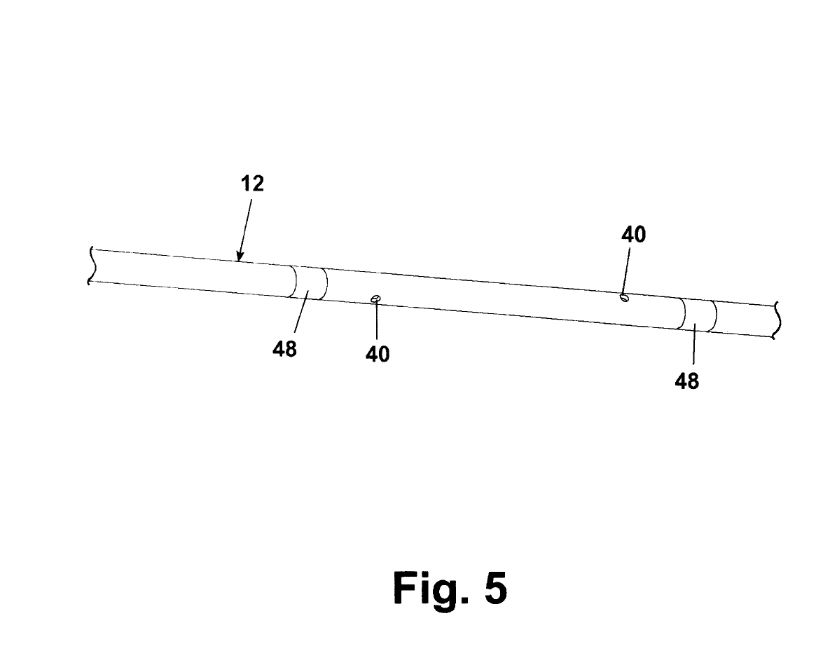

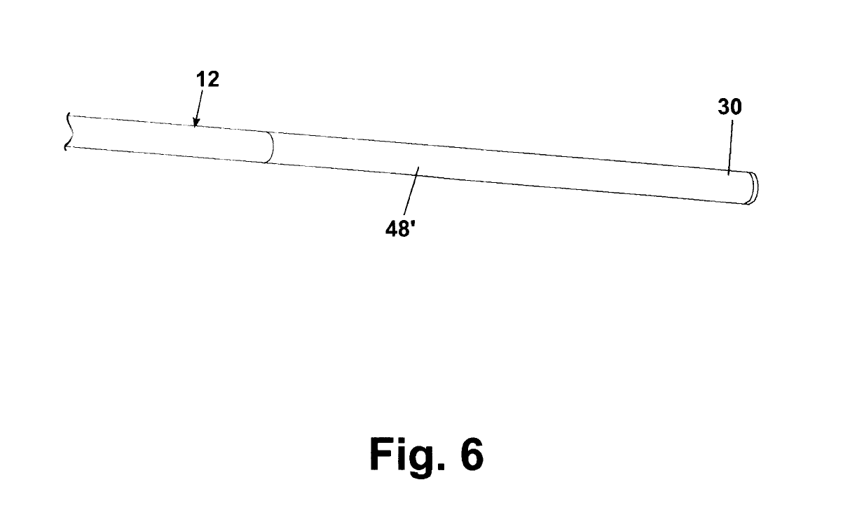

FIG. 5 shows a second alternative for the markers comprising a plurality of marker collars 48 encircling the tube 12 at preselected intervals. The marker collars 48 can comprise label-like elements that are fixedly attached, such as with a suitable adhesive, to the exterior of the tube 12. An alternative marker collar 48′ as shown in FIG. 6 is identical to the marker 48, except that it is an elongated collar encompassing the distal end 30 of the tube 12. In these preferred embodiments, an ink containing tungsten is used. Alternatively, the marker collars 48, 48′ can comprise a printed element which is printed with a tungsten ink directly on the exterior of the tube 12. The tungsten renders the markers echogenic, radiopaque, and MRI-compatible.

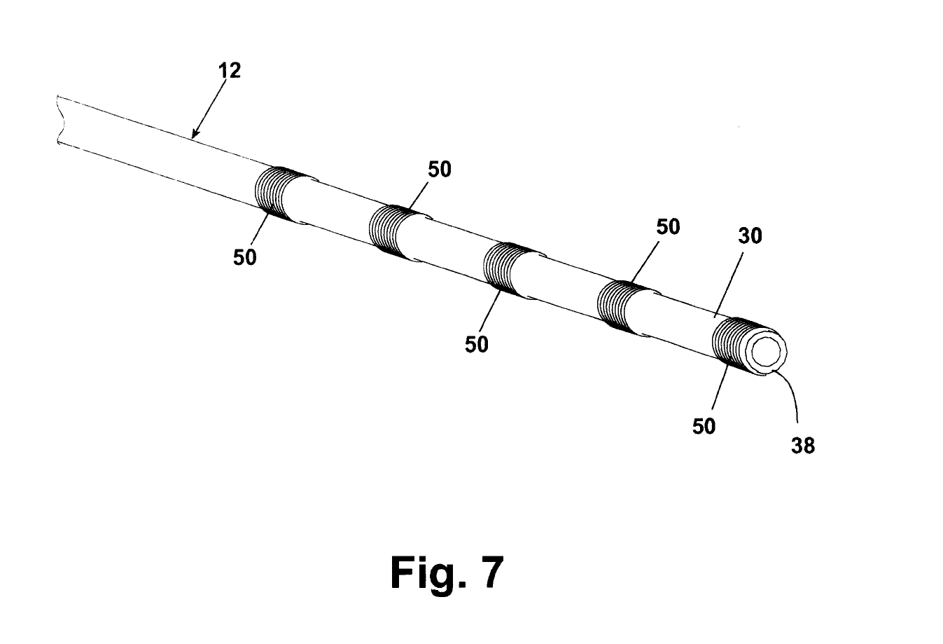

A third alternative for the markers is shown in FIG. 7. In this alternative, the markers comprise a plurality of external marker beads 50 encircling the tube 12 at preselected intervals. The marker beads 50 shown in FIG. 6 comprise helical coils which are fixedly attached to the exterior of the tube 12 through a suitable adhesive.

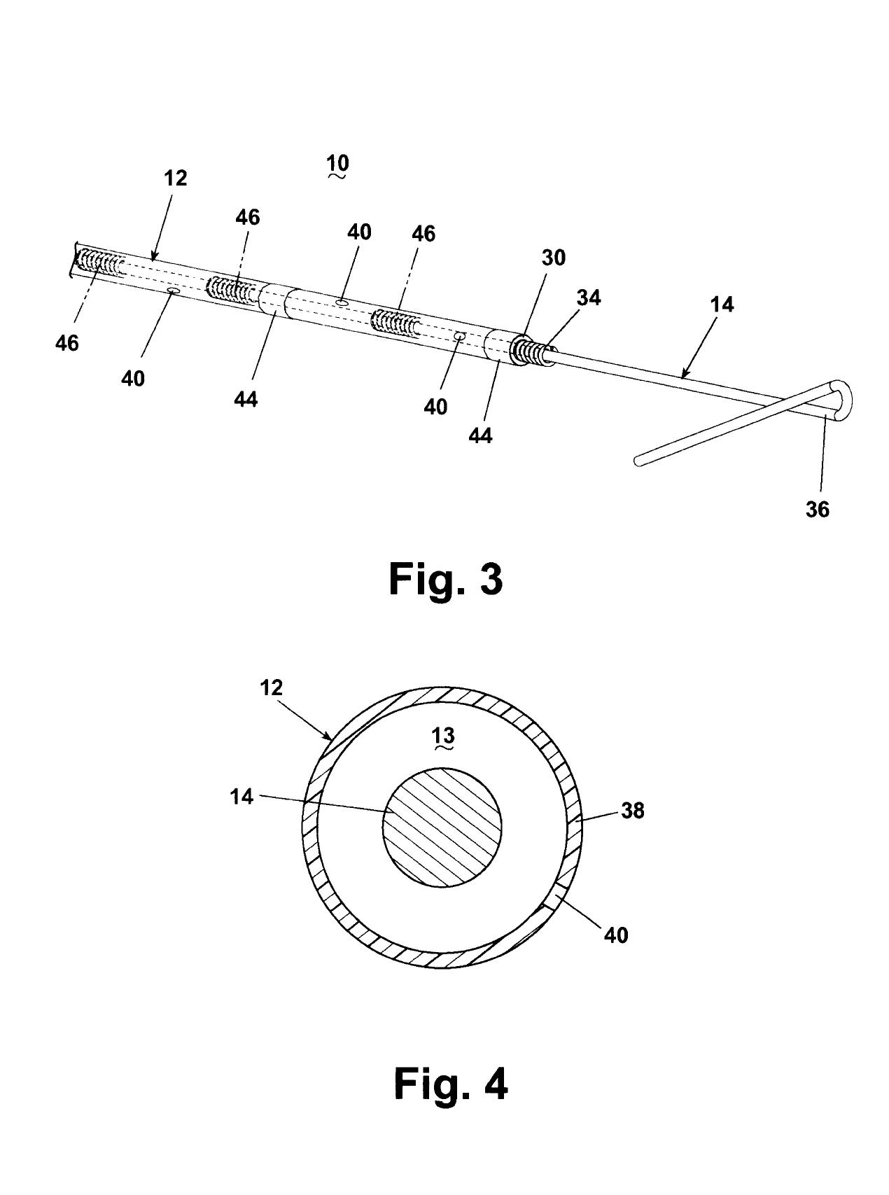

It will be readily apparent to one of ordinary skill in the art that the markers can be provided in any number and can comprise more than one type of marker as shown in FIG. 3 to facilitate the use of different imaging techniques.

Alternatively, the markers can be integrally formed with the wire. The integral structure can be in the form of the beads, for example. That is, a coil pattern formed on the wire. The coil pattern can extend from the wire or be formed in the wire, such as by forming a coil groove in the wire. Other patterns than a coil pattern can be used. The pattern can comprise a series of parallel and/or overlapping ribs and/or grooves. The advantage of mounting the markers to the wire or forming the markers with the wire is that the exterior of the conduit would be relatively smooth, which will reduce the degree of damage to the surrounding tissue upon the insertion and withdrawal of the needle from the tissue.

Multiple ports 40 are formed in the tube 12 and extending completely through the wall of the tube 12 to establish fluid communication between the duct 13 and the exterior of the tube 12. In this manner, fluid introduced into the tube 12 can travel through the duct 13 and exit the tube 12 through the ports 40.

The ports 40 are shown as circular openings that are located radially about the exterior of the tube 12. Preferably, there are ports 40 located at least every 90 degrees relative to the centerline of the tube 12. The number and location of the ports 40 can vary as needed.

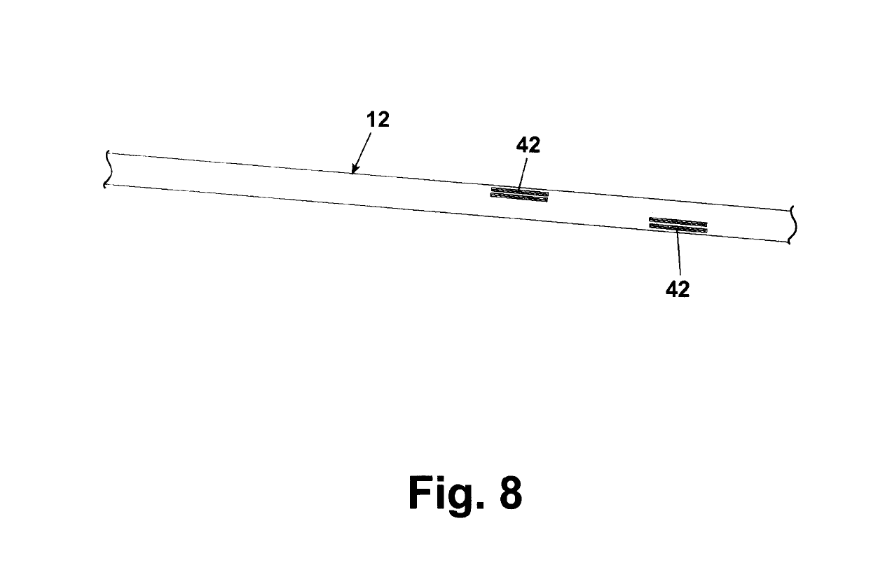

FIG. 8 shows an alternative structure for the infusion ports. In FIG. 8 the infusion tube 12 is provided with one or more radially-spaced infusion ports, in the form of slits 42 in fluid communication with the annular duct 13 for the injection of fluid through the annular duct 13 into the adjacent tissue. The slits can be spaced radially about the tube in the same fashion as the openings 40. Alternatively, the slits can be located at the distal end of the tube 12.

In yet another alternative, the infusion tube 12 can be fabricated with a porous or permeable section, preferably at the distal end 30, instead of apertures 40 or slits 42. The porosity of the permeable section can be selected to provide control of the infusion rates of the fluid into the surrounding tissue.

Referring to FIG. 1, the syringe assembly 28 is used for delivery of fluid into the annular duct 13. In the preferred embodiment, the syringe assembly comprises a generally conventional syringe 22 having a Luer-lock tip 23 that is connected in fluid-tight communication to a proximal end of a connector 24 having a mating Luer-lock receptacle 25 and comprising an axial bore longitudinally therethrough adapted for fluid communication with the syringe 22. A second, threaded end 27 of the connector 24 is adapted for fluid communication with the proximal end 32 of the infusion tube 12 and comprises a compression-type fitting having a threaded collar 26 which is threadably received on the distal end of the connector 24. The connector 24 is connected to the proximal end 32 by inserting the proximal end 32 into the connector 24 and tightening the threaded collar 26 onto the connector 24, thereby constricting the connector 24 around the infusion tube 22 to form a fluid-tight seal. An optional cap 29 is provided for closing the open end of the connector 24 when the syringe 22 is not connected. The benefit of the cap 29 is that it closes what would otherwise be an open conduit from the environment exteriorly to the body to the location of the infusion tube interiorly of the body to reduce the likelihood of infection or contamination, for example.

While the syringe assembly is the contemplated apparatus for introducing a diagnostic fluid into the infusion tube, any other suitable apparatus is within the scope of the invention since the particular apparatus is not limiting to the invention. For example, a suitable alternative includes connecting the infusion tube 12 to a fluid reservoir, such as an IV bag, for a generally continuous infusion of fluid into the tissue of interest. In yet another alternative, the infusion tube 12 can be fluidly connected to a reservoir containing a gas where an infusion of gas rather than fluid into the tissue of interest is desired.

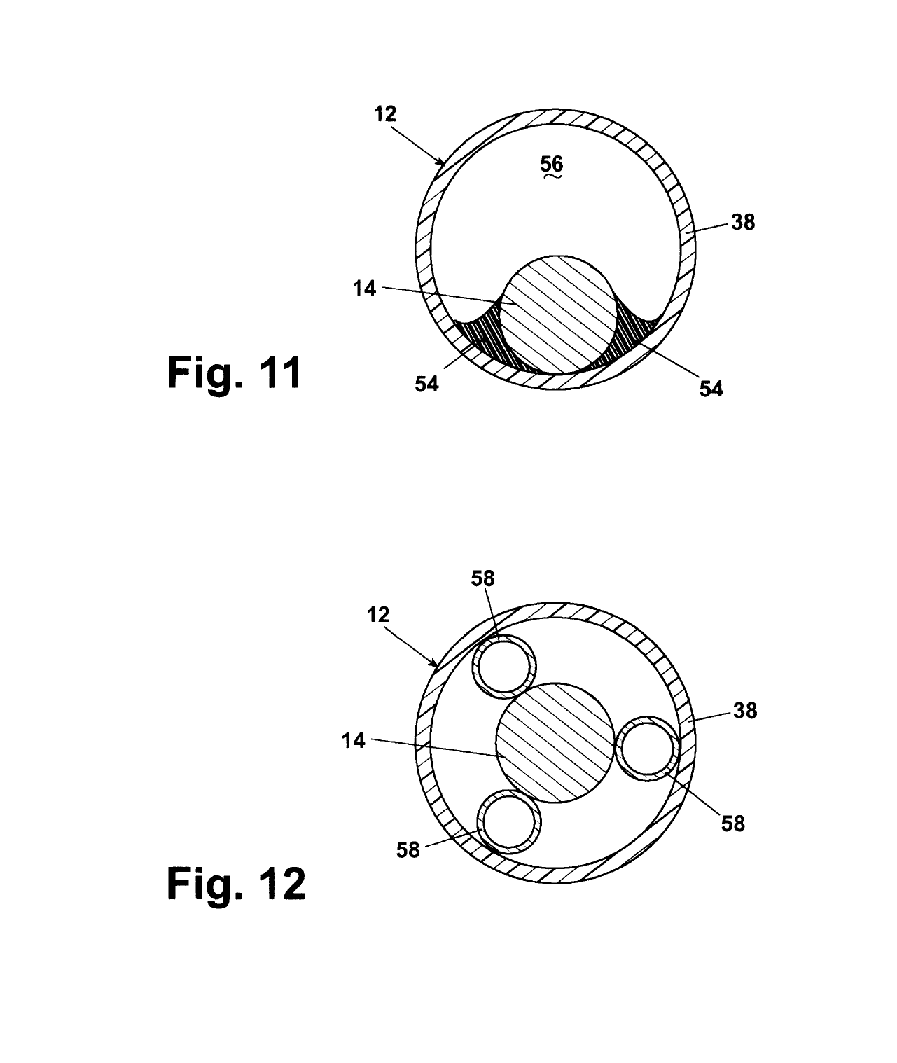

FIGS. 11 and 12 illustrate alternative mountings of the wire 14 to the tube 12. In FIG. 11, the wire 14 extends longitudinally along the wall 38 of the infusion tube 12 and is fixedly attached to the wall 38 through a suitable adhesive 54, thereby forming a non-annular duct 56 for delivery of fluid. In FIG. 12, the wire 14 extends coaxially along the infusion tube 12, but the annular duct 13 is provided with a plurality of lumens 58. The lumens 58 are adapted for selective delivery of different fluids. For example, a first lumen 58 can be utilized for the delivery of a dye or radioisotope, a second lumen 58 can be utilized for the delivery of a medication, and a third lumen 58 can be utilized for the delivery of an anesthetic. The lumens are preferably formed by tubes similar in construction, except smaller, than the tube 12.

The multiple lumen structure can also be used to enhance the imaging characteristics of the needle 10. For example, one or more of the multiple lumens could be filled with a material that was easily imagable using a particular imaging technique (x-ray, ultrasound, MRI, mammography, etc.). In the case of MRI, one or more of the lumens could be filled with gadolinium or similar material, which is highly imagable with an MRI.

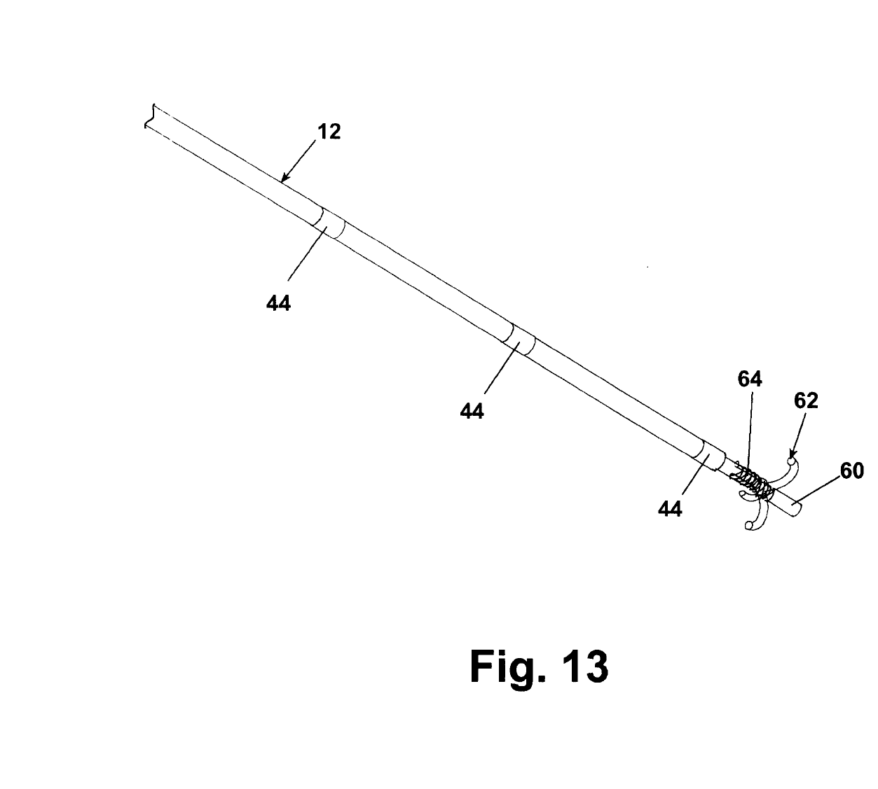

FIG. 13 shows a second embodiment of the localizing needle 10. The main difference between the second and first embodiments is the replacement of a fiber optic thread 60 for the wire 14, with most of the other elements being identical. Therefore, like numerals will be used to identify like parts and only the relevant differences will be described in detail.

In the second embodiment, the wire 14 is replaced with a generally conventional fiber optic thread 60 extending coaxially through the infusion tube 12. The fiber optic thread 60 is fixedly attached to the infusion tube 12 with an anchoring bead or tapered anchoring cone as described above. A hook 62 is attached to the fiber optic thread 60 between the end of the fiber optic thread 60 and the infusion tube 12 for anchoring the needle 10 while enabling light to be transmitted through the fiber optic thread 60. As shown in FIG. 13, the hook 62 is attached to the fiber optic thread 60 through a collar comprising a helical coil 64 which can be fixedly attached to the fiber optic thread 60 through a suitable adhesive.

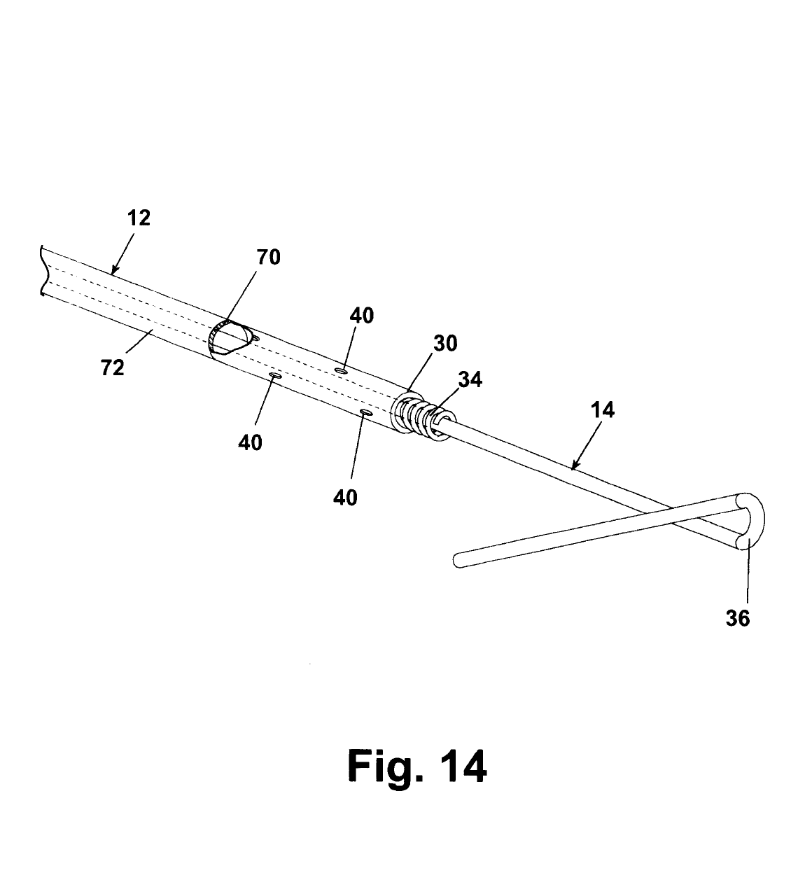

FIG. 14 shows an embodiment of the localizing needle 10 in which the infusion tube 12 comprises a separate infusion tip 68 attached to a non-perforated tube section 72 at their common interface 70. The infusion tip 68 comprises a section of tubing having infusion ports 40 to which a bead 34 is fixedly attached as heretofore described for fixedly attaching the wire 14 to the tube 12. The infusion tip 68 is attached to the tube section 72 through a suitable adhesive or other suitable method. In this way, different infusion tips having differing markers and infusion ports can be selectively fabricated, and attached to preselected lengths of conventional tubing to form the tube 12, thereby facilitating the fabrication of a variety of infusion tubes 12 while conserving materials.

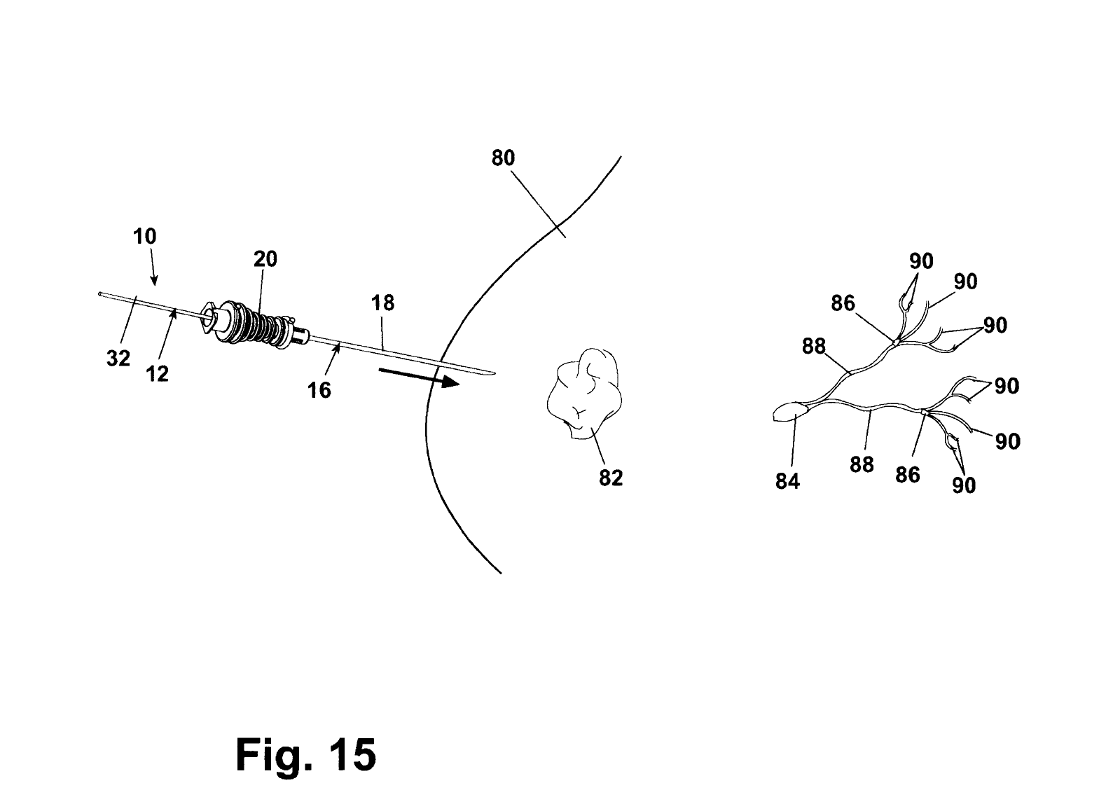

Referring to FIGS. 15-17, in use, the localizing needle 10 is inserted into the cannula 16 so that the distal end 30 and the barb 36 are within the insertion needle 18. Using an appropriate insertion device interconnected with the needle 18 through the hub 20, the cannula 16 with the enclosed localizing needle 10 is inserted into the patient to the desired location within the tissue of interest. The cannula 16 is then withdrawn, leaving the localizing needle 10 in place. As the cannula 16 is withdrawn, the barb 36 will be revealed, thereby releasing the anchor of the localizing needle 10 into the surrounding tissue.

A suitable imaging device is used to locate the localizing needle 10. The markers 44, 46, 48, and/or 50 are used in combination with the imaging device to properly locate the localizing needle 10.

Once the localizing needle 10 is properly placed, the syringe 22 can be fluidly connected to the infusion tube 12 through the connector 24 as described. A preselected fluid can then be delivered by the syringe into the infusion tube 12 and through the infusion ports 40, 42 to the tissue of interest.

The localizing needle 10 is ideally suited for locating the first or sentinel lymph node draining a particular region of a human breast in which a tissue mass, such as a tumor, is present. Locating the sentinel lymph node is very important in determining whether the cancer has traveled from the breast tissue to the lymphatic system as the sentinel node is the first lymph node that the cancer would travel to.

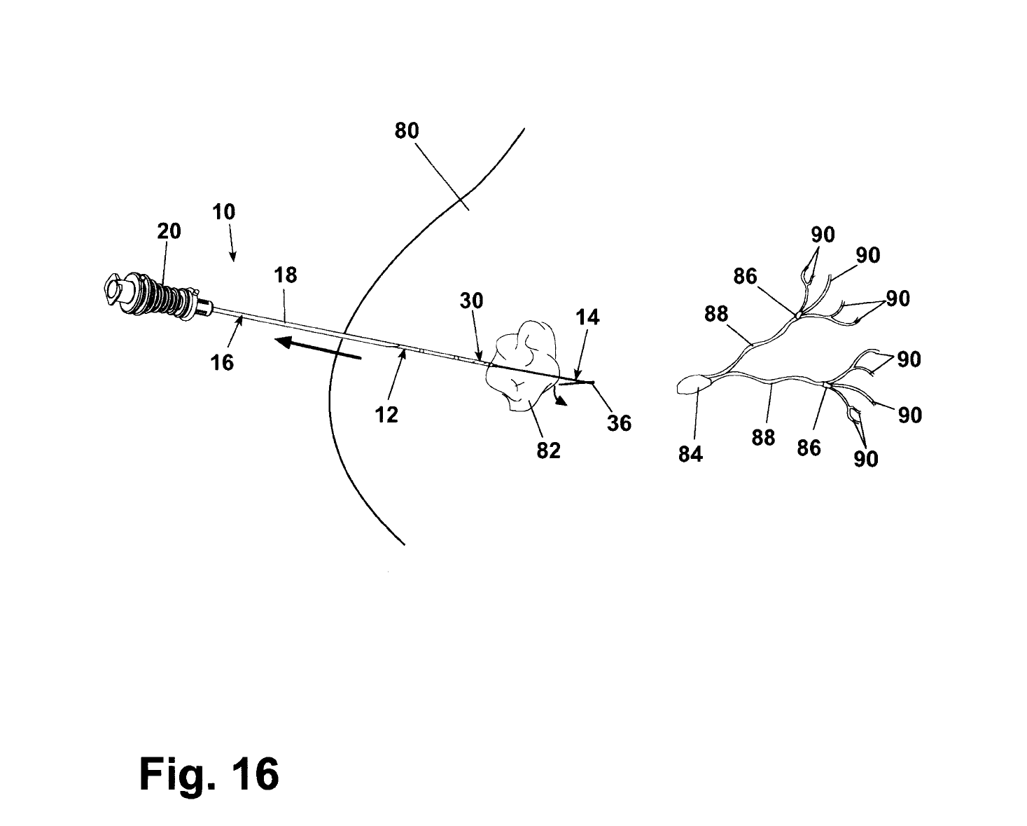

As shown in FIG. 15, in such a sentinel node procedure, the cannula 16 containing the localizing needle 10 is inserted into a breast 80 at the site containing a tumor 82. For illustrative purposes, the region containing the tumor 82 is shown as draining to a portion of the lymphatic system comprising a sentinel lymph node 84 connected by a sentinel duct 88 to a plurality of secondary lymph nodes 86 which, in turn, are drained by secondary ducts 90. When the localizing needle 10 is properly positioned, the cannula 16 is withdrawn, enabling the barb 36 to engage the breast tissue, leaving the localizing needle 10 anchored in place, as shown in FIG. 16. The markers aid in properly positioning the localizing needle 10. A suitable imaging device capable of imaging the markers is used to position the localizing needle 10.

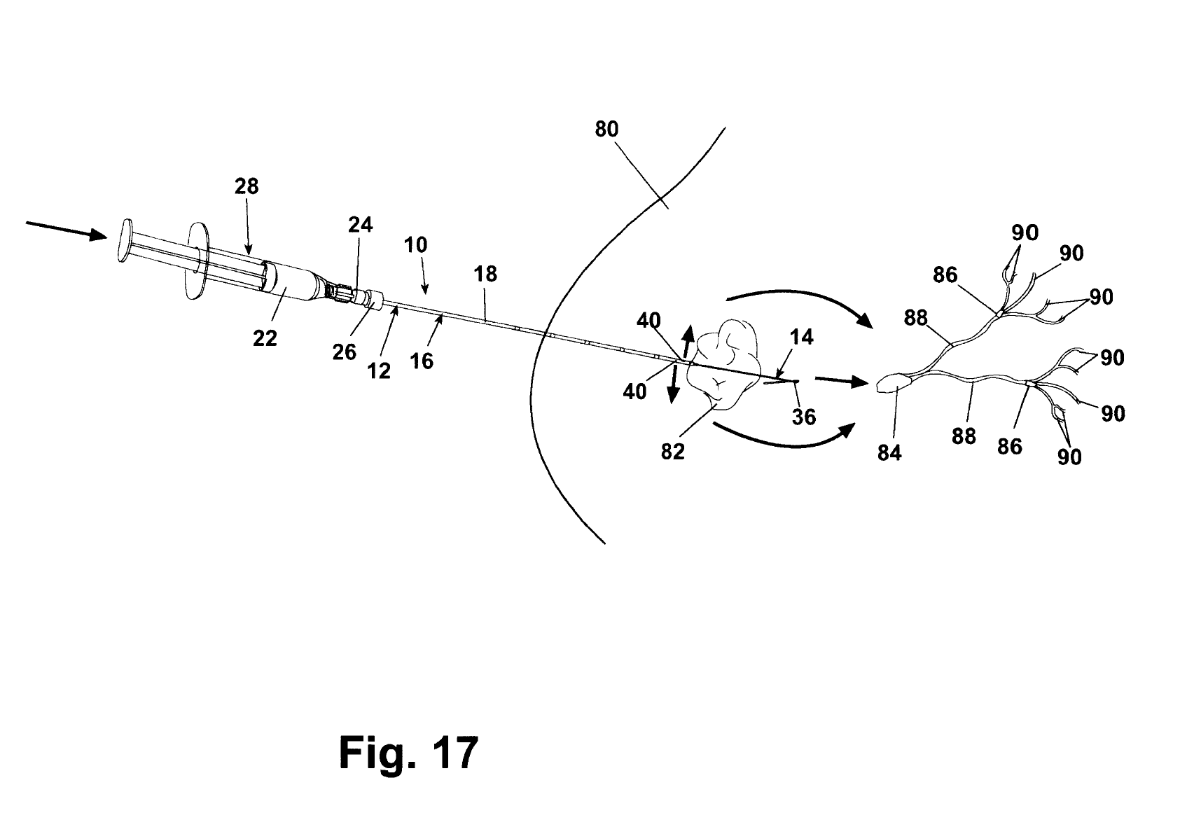

As shown in FIG. 17, a fluid, such as a dye, is introduced at the site of the tumor 82 through the tube 12 using the syringe assembly 28 connected to the tube 12, as previously described herein. The fluid will migrate to the lymph nodes 84, 86 draining the breast tissue, which can be monitored by a physician or other medical practitioner in the traditional manner. The sentinel node 84 is the first lymph node to which the dye migrates. Thus, the sentinel node 84 associated with the tumor 82 can be readily and accurately identified. A biopsy of the sentinel node 84 can then be taken to determine whether cancer has spread to the other lymph nodes 86.

By determining which lymph node is the sentinel node 84, the number of biopsies is greatly reduced. If the cancer has spread to the lymphatic system, it will first spread to the sentinel node 84. Thus, identifying the sentinel node 84 eliminates the need to biopsy all of the lymph nodes to determine if the cancer has spread from the breast 80 to the lymphatic system.

The invention has several advantages over prior art devices for injecting a fluid to determine the sentinel node. For example, the infusion tube is less costly to manufacture than hollowing the interior of a wire. It is generally easier to form the infusion tube since it is made from plastic than it is to drill out the interior of a solid wire. Similarly, it is easier to form the fluid ports in the hollow tube than it is to form them in the hollowed wire. The infusion also functions to protect the localizing wire from being cut during the excision of the biopsy, reducing the likelihood that localizing needle of the invention will be severed. The localizing wire of the invention is also stronger since it is solid, making it less likely that the wire will break during use. The infusion tube is also transparent or translucent, which provides the doctor with the ability to watch the fluid flow from the syringe and into the body. This will give the doctor some indication that the fluid is flowing properly from its storage container.

While the invention has been specifically described in connection with certain specific embodiments thereof, it is to be understood that this is by way of illustration and not of limitation, and the scope of the appended claims should be construed as broadly as the prior art will permit. For example, while it is contemplated that a dye or radioisotope will be injected through the infusion tube, any type of liquid or flowable material can be used. Similarly, while a Luer-lock is used to couple the syringe to the induction tube, any suitable connector can be used, including integrally forming the syringe with the tube.

1. A medical device for injecting fluid into a tissue at a selected location in the tissue, comprising:
an infusion tube having distal and proximal ends and defining a lumen with an outlet proximate the distal end and through which fluid in the lumen can exit from the infusion tube;
a localizing wire fixedly mounted to the infusion tube and comprising an anchor; and
wherein the medical device is positioned to deliver fluid to the selected location within the tissue by inserting the distal end of the infusion tube into the tissue such that fluid released through the outlet will flow to the selected location and the anchor engages the tissue to fix the position of the infusion tube.

2. The medical device of claim 1, and further comprising a retainer for fixedly coupling the localizing wire to the infusion tube.

3. The medical device of claim 2, wherein the retainer comprises an adhesive bonding the localizing wire to the infusion tube.

4. The medical device of claim 2, wherein the retainer comprises a bead encircling the localizing wire and received within the lumen.

5. The medical device of claim 4, wherein the bead is imagable with at least one of the following imaging methods: MRI, X-ray, Ultrasound, and Mammography.

6. The medical device of claim 2, wherein the retainer comprises the localizing wire being press-fit within the lumen.

7. The medical device of claim 2, wherein at least a portion of the localizing wire is received within the lumen.

8. The medical device of claim 7 wherein the outlet comprises at least one port extending from the lumen to the exterior of the infusion tube.

9. The medical device of claim 8 and further comprising multiple ports radially spaced about the infusion tube.

10. The medical device of claim 8 wherein the at least one port is formed by a generally circular opening formed in the infusion tube.

11. The medical device of claim 10 wherein the at least one port is formed by a slit formed in the infusion tube.

12. The medical device of claim 7 wherein the anchor is releasable to permit the repositioning of the infusion tube.

13. The medical device of claim 12 wherein the anchor comprises a flexible hook.

14. The medical device of claim 7 and further comprising at least one imaging marker provided on at least one of the infusion tube and anchor.

15. The medical device of claim 14 wherein there are multiple imaging markers.

16. The medical device of claim 15 wherein the imaging markers are spaced in a predetermined arrangement.

17. The medical device of claim 6 wherein the infusion tube further comprises an inlet for introducing fluid into the lumen.

18. The medical device of claim 17 and further comprising a syringe fluidly connected to the inlet to introduce fluid into the lumen from the syringe.

19. The medical device of claim 18 wherein the syringe is removably coupled to the inlet.

20. The medical device of claim 1 wherein the infusion tube comprises multiple, fluidly isolated lumens.

21. The medical device of claim 20 wherein at least a portion of one of the lumens is filled with a fluid that functions as an imaging marker to aid in the imaging of the medical device.

22. The medical device of claim 21 wherein the fluid is gadolinium.

23. The medical device of claim 1 wherein the localizing wire is mounted to the infusion tube proximate the distal end.

24. The medical device of claim 1 wherein the localizing wire extends through the lumen of the infusion tube.

25. The medical device of claim 24 wherein the localizing wire extends coaxially through the lumen.

26. The medical device of claim 24 wherein the infusion tube comprises multiple lumens and the wire extends through one of the multiple lumens.

27. The medical device of claim 24 wherein the wire comprises a repositionable hook forming the anchor.

28. The medical device of claim 1 wherein the localizing wire comprises a fiber optic thread.

29. The medical device of claim 28 wherein the anchor comprises a hook mounted to the fiber optic thread.

30. The medical device of claim 1 wherein the outlet comprises multiple ports extending through the infusion tube to establish fluid communication between the lumen and the exterior of the infusion tube.

31. The medical device of claim 30 wherein the ports are spaced radially about the infusion tube.

32. The medical device of claim 31 wherein the ports are arranged in at least one set, and the ports in the at least one set are at 90° relative to each other.

33. The medical device of claim 30 wherein the ports comprise circular apertures.

34. The medical device of claim 30 wherein the ports comprise slits.

35. The medical device of claim 1 and further comprising imaging markers provided on at least one of the infusion tube and the localizing wire.

36. The medical device of claim 35 wherein the imaging markers have at least one of the following characteristics: echogenic, radiopaque, magnetic resonance compatible.

37. The medical device of claim 35, wherein the imaging markers are imageable using at least one of the following methods: MRI, X-ray, Ultrasound, and Mammography.

38. The medical device of claim 35 wherein the imaging markers comprise helical coils.

39. The medical device of claim 38 wherein the helical coils are located in the lumen.

40. The medical device of claim 38 wherein the helical coils encircle the infusion tube.

41. The medical device of claim 35 wherein the imaging markers comprise a label imprinted with ink.

42. The medical device of claim 41 wherein the ink comprises tungsten.

43. The medical device of claim 1 and further comprising a cannula for locating the infusion tube into the tissue, the cannula having a lumen sized to receive the infusion tube and localizing wire, wherein the infusion tube and localizing wire are received within the cannula lumen, the cannula is inserted into the tissue, and a portion of the infusion tube is exposed to the tissue from the cannula.

44. The medical device of claim 43 and further comprising a syringe fluidly coupled to the infusion tube lumen for the delivery of the fluid to the infusion tube.

45. The medical device of claim 44 and further comprising a releasable connector for releasably connecting the syringe to the infusion tube.

46. The medical device of claim 45 wherein the releasable connector is a Luer-lock connector.

47. The medical device of claim 46 wherein the releasable connector is connected to the infusion tube with a compression fitting.

48. The medical device of claim 47 wherein the infusion is transparent or translucent to permit the exterior viewing of the fluid as it flows through the lumen.

49. A method of injecting a fluid into a tissue mass using a localizing needle comprising an infusion tube defining a lumen, a localizing wire fixedly mounted to the infusion tube, and at least one imaging marker located on the infusion tube or localizing wire, comprising the steps of:
inserting the infusion tube and localizing wire into tissue mass;
locating the infusion tube at a predetermined location within the tissue mass by imaging the imaging marker;
anchoring the localizing wire at the predetermined location to fix the position of the infusion tube within the tissue mass, and
delivering a fluid to the tissue mass through the lumen.

50. The method of claim 49 wherein the locating of the infusion tube comprises locating the infusion tube near a biopsy site in the tissue mass.

51. The method of claim 50 wherein the delivering of fluid comprises delivering fluid through at least one port in the infusion tube.

52. The method of claim 51 wherein the infusion tube is located near the biopsy site such that the fluid exiting the at least one port flows to the biopsy site.

53. The method of claim 52 and further comprising the step of detecting the movement of the fluid from the biopsy site to another location.

54. The method of claim 53 wherein the biopsy site is located within a human breast and the another location is a node of the human lymphatic system.

55. The method of claim 54 wherein the inserting step comprises inserting the infusion tube and localizing wire into a cannula.

56. The method of claim 55 wherein the cannula is inserted into the tissue prior to the insertion of the infusion tube and localizing wire into the cannula.

57. The method of claim 56 wherein the cannula is withdrawn from the tissue mass after the locating of the infusion tube.

58. The method of claim 49 and further comprising the step of detecting the movement of the fluid to a second tissue mass.

59. The method of claim 58 wherein the tissue mass is a human breast and the second tissue mass is a node of the human lymphatic system.
